(19) United States
(12) Patent Application Publication
(10) Pub. No.: US 2025/0205271 A1
(43) Pub. Date: Jun. 26, 2025

(54) MICRORNAS FOR INHIBITING THE EXPRESSION OF TRF2 IN TUMORS

(71) Applicants: ISTITUTI FISIOTERAPICI OSPITALIERI (IFO), Roma (IT); UNIVERSITA' DEGLI STUDI DI TRIESTE, Trieste (IT)

(72) Inventors: Annamaria BIROCCIO, Roma (IT); Stefan SCHOEFTNER, Trieste (IT); Roberto DINAMI, Roma (I); Eleonora PETTI, Roma (IT); Luca POMPILI, Roma (IT)

(73) Assignees: ISTITUTI FISIOTERAPICI OSPITALIERI (IFO), Roma, RM (IT); UNIVERSITA' DEGLI STUDI DI TRIESTE, Trieste, TS (IT)

(21) Appl. No.: 18/850,191

(22) PCT Filed: Mar. 23, 2023
(86) PCT No.: PCT/IT2023/050088

§ 371 Date: Sep. 24, 2024

(30) Foreign Application Priority Data
Mar. 24, 2022 (IT) 102022000005786

(51) Int. Cl. A61K 31/7105; A61K 9/51; A61K 45/06; A61P 35/04; C12Q 1/6886

(52) U.S. Cl. CPC A61K 31/7105; A61K 9/5107; A61K 45/06; A61P 35/04; C12Q 1/6886

(57) ABSTRACT

The present invention relates to microRNA for use in the treatment of tumours, such as, for example, triple negative breast cancer, by inhibition of the expression of TRF2.

The present invention relates to microRNA for inhibiting the expression of TRF2 in tumours.

In particular, the invention relates to hsa-miR-182-3p for use in the treatment of tumours, such as for example triple negative breast cancer, through the inhibition of TRF2 expression.

It is well known that many types of tumours overexpress TRF2 compared to healthy tissues (Cherfils-Vicini et al. 2019), including colon cancer (Biroccio et al. 2013), lung cancer (Nakanishi et al. 2003) and mouth cancer (Benhamou et al. 2016).

In these tumours TRF2, a subunit of the Shelterin complex which is fundamental for telomere replication and maintenance, acts by favouring carcinogenesis.

In particular, it is known that breast cancer expresses high TRF2 levels, compared to the normal counterpart, and that a higher expression of TRF2 is correlated to a worse prognosis of this type of tumour (Cherfils-Vicini et al. 2019).

Triple negative breast cancer (TNBC) represents about 20% of the cases of breast cancer. TNBC is a particularly aggressive subtype, with a relative five-year survival rate of about 10%, and there are no specific treatments for it yet. In fact, its molecular characteristics currently make it difficult to identify appropriate therapies. Specifically, the absence of oestrogen and progesterone receptors precludes the use of hormone therapies, whilst the absence of HER2 also precludes the use of so-called molecular targeted therapy, a standard treatment for patients with breast cancers that are positive for the latter receptor. Negativity to these three tumour markers thus reduces the treatment options, limiting them mostly to classic chemotherapy.

Moreover, this type of tumour has a high rate of recurrence at a local and/or metastatic level. In particular, in patients with distant metastasis, global survival is about 12 months, also given the scarce effectiveness of standard therapies, and there is thus an increasingly pressing need to find new specific drugs (Yin et al. 2020).

Progress in the treatment of patients with triple negative breast cancer has led the European Medicines Agency (EMA) to approve the immunoconjugate sacituzumab govitecan, thanks to the first promising results (Bardia et al. 2019). For the time being, it has been approved for use only as a third-line treatment for inoperable and/or metastatic tumours. However, this drug has adverse effects (including myelotoxic effects), so there are still no effective molecular targeted therapies for TNBC.

In the light of the above, it is apparent that there is a need to provide new therapies for tumours overexpressing TRF2, particularly breast cancer and, more particularly, triple negative breast cancer, which are capable of overcoming the disadvantages of the known therapies.

The solution according to the present invention fits into this context; it aims to provide new molecular targeted antitumour drugs against tumours overexpressing TRF2.

In particular, according to the present invention it has now been surprisingly found that miR-182-3p, miR-519e-5p and miR-296-3p are efficient in modulating the expression of the protein TRF2.

More in particular, as shown in the experimental data reported further below, when the effects caused by the modulation of the telomeric protein TRF2 were studied, miR-182-3p was identified as a specific regulator of TRF2.

Based on the results obtained according to the present invention, miR-182-3p has shown anti-proliferative properties in multiple tumour models, including cervical cancer, colon cancer, osteosarcoma, pancreatic cancer, glioblastoma, mouth cancer and triple negative breast cancer. In particular, as regards the tested models of triple negative breast cancer, they include both stabilized tumour cells and advanced preclinical models, such as the advanced in vitro model of cell cultures created from implants of patient-derived triple negative breast tumours (PDTCs). Promising results have also been obtained in resistant triple negative breast cancer models.

Moreover, according to the present invention, the microRNA was encapsulated in lipid nanoparticles as a delivery system, making it possible to test the treatment based on miR-182-3p activity in several preclinical models, such as patient-derived tumour xenografts (PDX), among the most predictive of the therapeutic response, thereby confirming the anti-TRF2 activity and showing its antitumour effect.

The results obtained in vitro and in vivo according to the present invention consistently showed the antitumour activity of miR-182-3p associated with effective regulation of TRF2 levels.

According to the present invention, a drug capable of reducing the intratumour expression of TRF2, with evident antitumour properties, is being proposed for the first time. In fact, though the pro-tumourigenic properties of TRF2 have been known for some time, to date no drug has been developed ex novo to inhibit its activity or reduce its expression.

Moreover, since the unique microvascularization properties of the central nervous system represent a constant difficulty for the effective distribution of drugs to brain tissue, the effectiveness of LNPs-miR-182-3p observed in the intracranial implant model according to the present invention represents a relevant finding regarding the potential of applying treatment with LNPs-miR-182-3p in primary or metastatic brains tumours.

Therefore, according to the present invention, an anti-TRF2 therapy based on the action of microRNA is provided for the first time.

As is well known, microRNAs (miRNAs) are small non-encoding RNA molecules of 21-24 nucleotides which perform a crucial role in regulating gene expression through the inhibition of protein synthesis, mainly by interacting with the 3′ untranslated region (3′ UTR) of the target mRNAs (O'Brien et al. 2018). Thanks to progress made in nucleic acid delivery systems, miRNAs represent an important tool for the development of new generation antitumour therapies (Wong et al. 2020). Preclinical study of the activity of these molecules has developed thanks to the use of lipid nanoparticles (Lee et al. 2019), a carrier system that has been used for a number of years now and which enables molecules to be stabilized and delivered into the bloodstream, while preventing their degradation (Müller et al., 2000). Moreover, their lipidic nature represents an advantage for the process of intracellular absorption, of particular interest for tumours in which the change in the composition of the plasma membrane results in a decrease in permeability to common chemotherapeutics (Rivel et al. 2019). For some time, therefore, studies have been carried out on the effectiveness of new nucleic acids encapsulated in lipid carriers (Di Martino et al. 2014). The approval of the first clinical antitumour experimentation with a regulatory RNA in 2015 is encouraging (Ozcan et al. 2015).

According to the present invention, it has been observed, moreover, that the expression of TRF2 is correlated with the formation of metastasis in breast cancer, such as triple negative breast cancer.

Therefore, the worst prognosis associated with TRF2 expression in breast cancer is correlated with metastasis formation. This correlation suggests the possibility of considering TRF2 as a new marker in the classification of breast tumours, particularly in the identification of breast tumours with a higher risk of developing metastasis.

It is therefore a specific object of the present invention miRNA hsa-miR-182-3p (hsa-miR-182*, MIMAT0000260) for use in the treatment of a tumour overexpressing TRF2, said tumour overexpressing TRF2 being a tumour wherein the expression of TRF2 is greater compared to the expression of TRF2 in a healthy tissue.

According to one embodiment of the present invention, said tumour overexpressing TRF2 is not osteosarcoma. Moreover, according to a further embodiment, said tumour overexpressing TRF2 is not neuroblastoma.

According to one aspect, the present invention also relates to one or more miRNAs selected from hsa-miR-182-3p (hsa-miR-182*, MIMAT0000260), hsa-miR-296-3p (MIMAT0004679) and hsa-miR-519e-5p (MIMAT0002828), preferably hsa-miR-182-3p, for use in the treatment of tumours overexpressing TRF2.

Said one or more miRNAs can be used as is or they can comprise specific modifications capable of improving their stability and increasing their affinity for binding with their target. For example, some of the chemical modifications that can be used are: phosphodiester bonds, 2′-O-(2-methoxyethyl) indicated as O_MOE, 2′-O-methyl (OMe), 2′-locked nucleic acid (LNA) and 2′-fluorine.

Said one or more miRNAs according to the present invention are preferably double-stranded miRNAs.

As mentioned above, according to the present invention, tumours overexpressing TRF2 means tumours wherein TRF2 is overexpressed, i.e. it is more greatly expressed compared to its expression in a healthy tissue.

According to the present invention, said one or more miRNAs can be used on their own or in combination with one another. In particular, according to the present invention, when said miRNAs are used in combination, they can be selected from hsa-miR-182-3p and hsa-miR-296-3p; hsa-miR-182-3p and hsa-miR-519e-5p; hsa-miR-296-3p and hsa-miR-519e-5p; or hsa-miR-182-3p, hsa-miR-296-3p and hsa-miR-519e-5p.

According to the present invention, said tumour can be selected from breast cancer, cervical cancer, colon cancer, osteosarcoma, primary brain tumours, such as, for example, glioblastoma, metastatic brain tumours, pancreatic cancer, and head and neck cancer. In particular, said breast cancer can be a triple negative breast cancer, for example a triple negative breast cancer mutated in BRCA1, optionally resistant to PARP inhibitors, or HER2-positive breast cancer.

According to the present invention, said one or more miRNAs can be encapsulated in lipid nanoparticles.

According to the present invention, said lipid nanoparticles are lipid-based particles capable of accommodating larger negatively charged molecules such as nucleic acids. A well-known example in clinical medicine is ‘patisiran’ lipid nanoparticles (ONPATTRO). [Adams, D. et al. Patisiran, an RNAi therapeutic, for hereditary transthyretin amyloidosis. N. Engl. J. Med. 379, 11-21 (2018)].

The present invention also relates to a pharmaceutical composition comprising miRNA hsa-miR-182-3p, together with one or more pharmaceutically acceptable excipients and/or adjuvants, for use in the treatment of a tumour overexpressing TRF2, said tumour overexpressing TRF2 being a tumour wherein the expression of TRF2 is greater compared to the expression of TRF2 in a healthy tissue.

As mentioned above, according to one embodiment of the present invention, said tumour overexpressing TRF2 is not osteosarcoma and is not neuroblastoma.

According to one aspect, the present invention also relates to a pharmaceutical composition comprising one or more miRNAs selected from hsa-miR-182-3p hsa-miR-296-3p and hsa-miR-519e-5p, preferably hsa-miR-182-3p, together with one or more pharmaceutically acceptable excipients and/or adjuvants, for use in the treatment of tumours overexpressing TRF2.

In the pharmaceutical composition according to the present invention, said one or more miRNAs can be present on their own or in combination with one another. In particular, according to the present invention, when said miRNAs are used in combination, they can be selected from hsa-miR-182-3p and hsa-miR-296-3p; hsa-miR-182-3p and hsa-miR-519e-5p; hsa-miR-296-3p and hsa-miR-519e-5p; or hsa-miR-182-3p, hsa-miR-296-3p and hsa-miR-519e-5p.

As mentioned above, according to the present invention, said tumour can be selected from breast cancer, cervical cancer, colon cancer, osteosarcoma, primary brain tumours, such as, for example, glioblastoma, metastatic brain tumours, pancreatic cancer, head and neck cancer. In particular, said breast cancer can be a triple negative breast cancer, for example a triple negative breast cancer mutated in BRCA1, optionally resistant to PARP inhibitors, or HER2-positive breast cancer.

In the pharmaceutical composition according to the present invention, said one or more miRNAs can be encapsulated in lipid nanoparticles. As mentioned above, according to the present invention, said lipid nanoparticles are lipid-based particles capable of accommodating larger negatively charged molecules such as nucleic acids.

According to the present invention, said pharmaceutical composition can further comprise one or more antitumour drugs, such as, for example, platinum derivatives, such as Cisplatin, taxanes, such as Paclitaxel, pyrimidine analogues, such as Gemcitabine, anthracyclines, such as Doxorubicin, immune checkpoint inhibitors, such as Atezolizumab, PARP1 inhibitors, such as Olaparib, and antibody-drug conjugates such as Sacituzumab-Govitecan.

Moreover, the present invention relates to a combination of hsa-miR-182-3p with one or more antitumour drugs, such as, for example, platinum derivatives, such as Cisplatin, taxanes, such as Paclitaxel, pyrimidine analogues, such as Gemcitabine, anthracyclines, such as Doxorubicin, and immune checkpoint inhibitors, such as Atezolizumab, PARP1 inhibitors, such as Olaparib, and antibody-drug conjugates such as Sacituzumab-Govitecan, for separate or sequential use in the treatment of a tumour overexpressing TRF2, said tumour overexpressing TRF2 being a tumour wherein the expression of TRF2 is greater compared to the expression of TRF2 in a healthy tissue.

As mentioned above, according to one embodiment of the present invention, said tumour overexpressing TRF2 is not osteosarcoma and is not neuroblastoma.

According to one aspect, the present invention further relates to a combination of one or more miRNAs selected from hsa-miR-182-3p, hsa-miR-296-3p and hsa-miR-519e-5p, preferably hsa-miR-182-3p, with one or more antitumour drugs, such as, for example, platinum derivatives, such as Cisplatin, taxanes, such as Paclitaxel, pyrimidine analogues, such as Gemcitabine, anthracyclines, such as Doxorubicin, immune checkpoint inhibitors, such as Atezolizumab, PARP1 inhibitors, such as Olaparib, and antibody-drug conjugates such as Sacituzumab-Govitecan, for separate or sequential use in the treatment of tumours overexpressing TRF2.

According to the present invention, separate use means administration, at the same time, of the compounds of the combination according to the invention in distinct pharmaceutical forms.

According to the present invention, sequential use means successive administration of the compounds of the combination according to the invention in a distinct pharmaceutical form.

According to the combination of the present invention of one or more miRNAs selected from hsa-miR-182-3p, hsa-miR-296-3p and hsa-miR-519e-5p3p with one or more antitumour drugs, said one or more miRNAs can be selected from hsa-miR-182-3p; hsa-miR-296-3p; hsa-miR-519e-5p; hsa-miR-182-3p and hsa-miR-296-3p; hsa-miR-182-3p and hsa-miR-519e-5p; hsa-miR-296-3p and hsa-miR-519e-5p; or hsa-miR-182-3p, hsa-miR-296-3p and hsa-miR-519e-5p.

According to the combination of the present invention, said tumour can be selected from breast cancer, cervical cancer, colon cancer, osteosarcoma, primary brain tumours, such as, for example, glioblastoma, metastatic brain tumours, pancreatic cancer, and head and neck cancer. In particular, said breast cancer can be a triple negative breast cancer, for example a triple negative breast cancer mutated in BRCA1, optionally resistant to PARP inhibitors, or HER2-positive breast cancer.

Moreover, according to the combination of the invention, said one or more miRNAs can be encapsulated in lipid nanoparticles. As mentioned above, according to the present invention, said lipid nanoparticles are lipid-based particles capable of accommodating larger negatively charged molecules such as nucleic acids.

Therefore, the miRNAs according to the present invention, in particular miR-182-3p, can be used, in the forms described above, and in the tumours specified above, in a method for treating a tumour overexpressing TRF2, i.e. a tumour wherein the expression of TRF2 is greater than its expression in a healthy tissue, said method comprising
administering to a patient having a tumour overexpressing TRF2 one or more miRNAs selected from hsa-miR-182-3p, hsa-miR-296-3p and hsa-miR-519e-5p, preferably hsa-miR-182-3p, or a pharmaceutical composition comprising said one or more miRNAs selected from hsa-miR-182-3p, hsa-miR-296-3p and hsa-miR-519e-5p, preferably hsa-miR-182-3p, together with one or more pharmaceutically acceptable excipients and/or adjuvants.

A further object of the present invention is an in vitro method for assessing the risk of developing metastasis in a patient with primary breast cancer, said method comprising measuring the expression of TRF2 in a sample of primary breast cancer, in particular a biopsy of primary breast cancer,
wherein an expression of TRF2 higher than, for example at least double, the expression of TRF2 in a sample of healthy tissue and/or in a sample of non-metastatic primary breast cancer, indicates a risk of developing metastasis.

The present invention also relates to a method for diagnosing and treating breast tumours with a risk of metastasis, said method comprising
a) obtaining a measurement of TRF2 expression in a primary breast cancer sample of a patient;
b) identifying a patient with primary breast cancer at risk of metastasis,
wherein the expression of TRF2 is higher than, for example at least double, the expression of TRF2 in a sample of healthy tissue and/or in a sample of non-metastatic primary breast cancer;
c) treating said patient with an adjuvant therapy, also in the presence of favourable clinical-pathological features.

In the event of occurrence of metastases, including brain metastases, the method of the invention can comprise administering one or more miRNAs selected from hsa-miR-182-3p, hsa-miR-296-3p and hsa-miR-519e-5p, preferably hsa-miR-182-3p, in the forms described above, or a pharmaceutical composition comprising said one or more miRNAs together with one or more pharmaceutically acceptable excipients and/or adjuvants.

The present invention will now be described by way of non-limiting illustration according to a preferred embodiment thereof, with particular reference to the examples and the figures in the appended drawings, wherein:

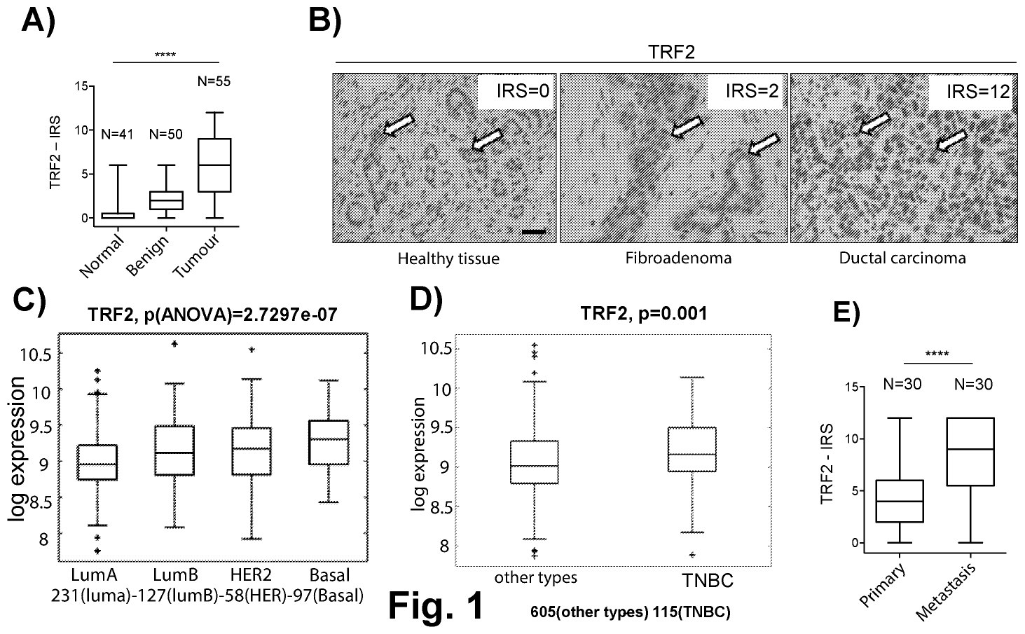

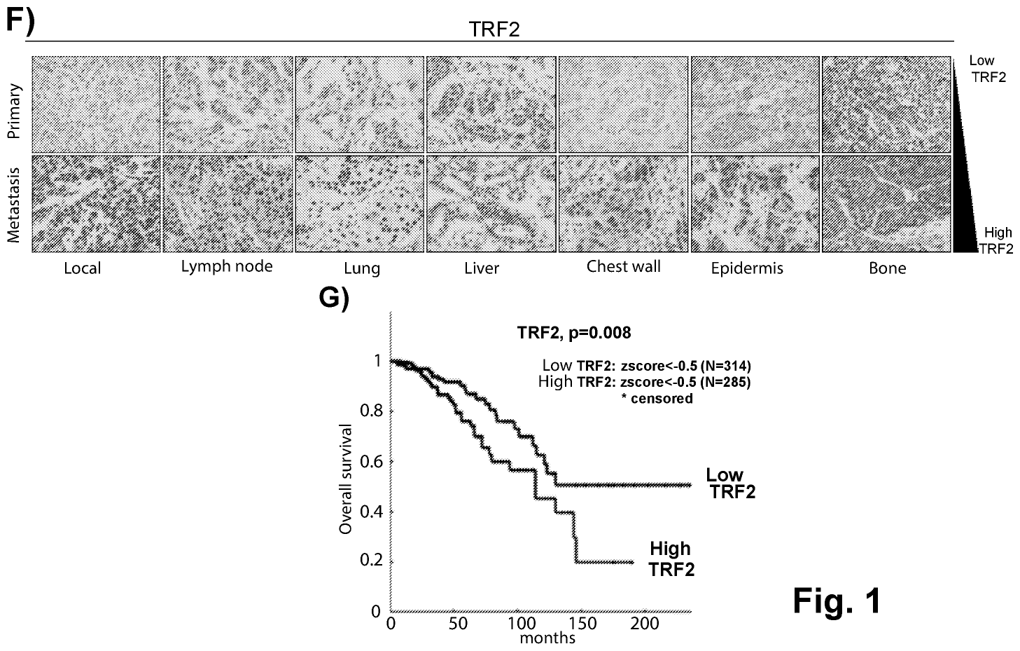

FIG. 1 shows that the expression of TRF2 correlates positively with breast cancer progression and metastasization. A. Quantification of TRF2 expression (Immunoreactive score-IRS) in healthy breast tissues (N=41), benign lesions (N=50) and malignant tumours (N=55) surgically treated at the Regina Elena National Cancer Institute. Statistical significance calculated by one-way ANOVA (**** P<0.0001). B. Images representative of immunohistochemical analysis on healthy tissue, fibroadenoma (benign lesion) and ductal carcinoma (malignant tumour). Scale bars, 30 μm. C. Analysis of TRF2 mRNA expression in breast cancer patients, from the TCGA dataset, stratified on the basis of the molecular subtype. Luminal A, N=231; Luminal B, N=127; HER2+, N=58; Basal, N=97. Statistical significance calculated by one-way ANOVA (**** P<0.0001). D. Analysis of TRF2 mRNA expression in breast cancer patients, from the TCGA dataset, stratified on the basis of the presence of oestrogen and progesterone receptors and HER2. Patients with positivity for at least one receptor (Others), N=605. Patients negative for all three receptors (TNBC), N=115. Statistical significance calculated by one-way ANOVA (*P<0.05). E. Quantification of TRF2 expression (IRS) in primary TN breast cancer and autologous metastatic lesions in patients from the Regina Elena National Cancer Institute (N=30). Statistical significance calculated by means of the Wilcoxon matched-pairs rank test (**** P<0.0001). F. Images representative of immunohistochemical analysis of TRF2 expression in primary TN breast cancer and corresponding metastatic lesions (local recurrence and recurrence in various organs distant from the primary site). G. Overall survival evaluated with a Kaplan-Meier curve in breast cancer cases drawn from the TCGA. The patients were stratified on the basis of TRF2 mRNA expression. Statistical significance calculated by means of the log-rank test (*P<0.05).

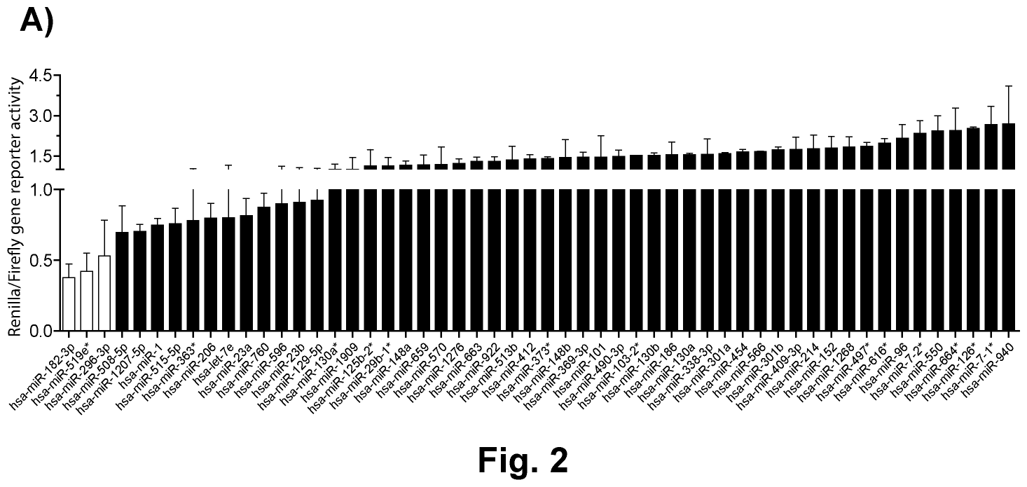

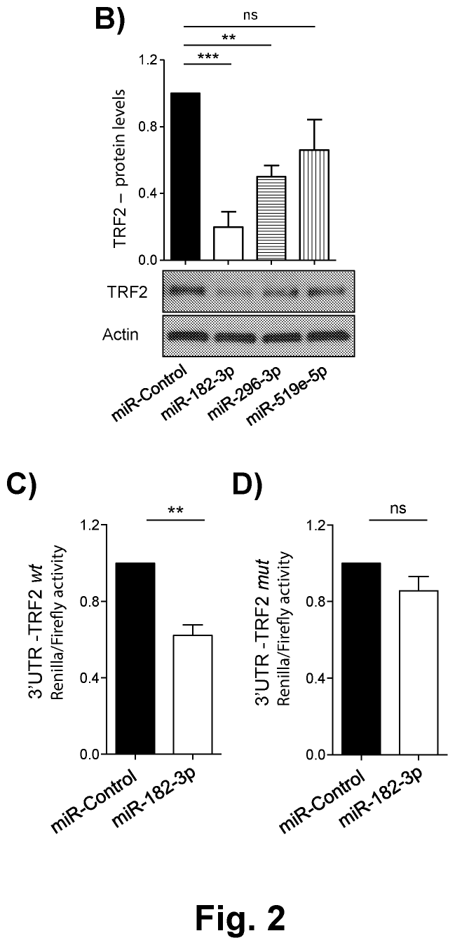

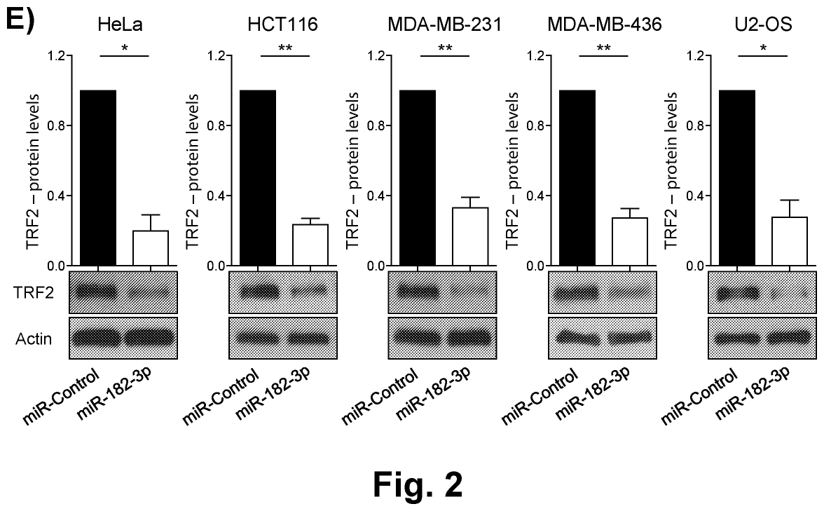

FIG. 2 shows the identification of miR-182-3p as a regulator of TRF2 expression. A. Results of high-throughput screening based on the Renilla luciferase reporter assay. The data show the Renilla luciferase reporter ratio for each candidate miRNA. The reporter ratio of a control miRNA was set on “1”. Reporter ratios <1 indicate the specific target of miRNAs which are candidates for the 3′UTR of TRF2. B. Western blot in Hela cells transfected with the miRNAs indicated (miR-Control, miR-182-3p, miR-519e-5p, miR-296-3p). Top panel: quantification of TRF2 levels. Bottom panel: representative image. Luciferase reporter assay in HeLa using the 3′-UTR of TRF2, wild type (wt) (C) or the mutated construct (mut) in the target site of miR-182-3p (D). E. Western blot on TRF2 in a panel of human tumour lines.

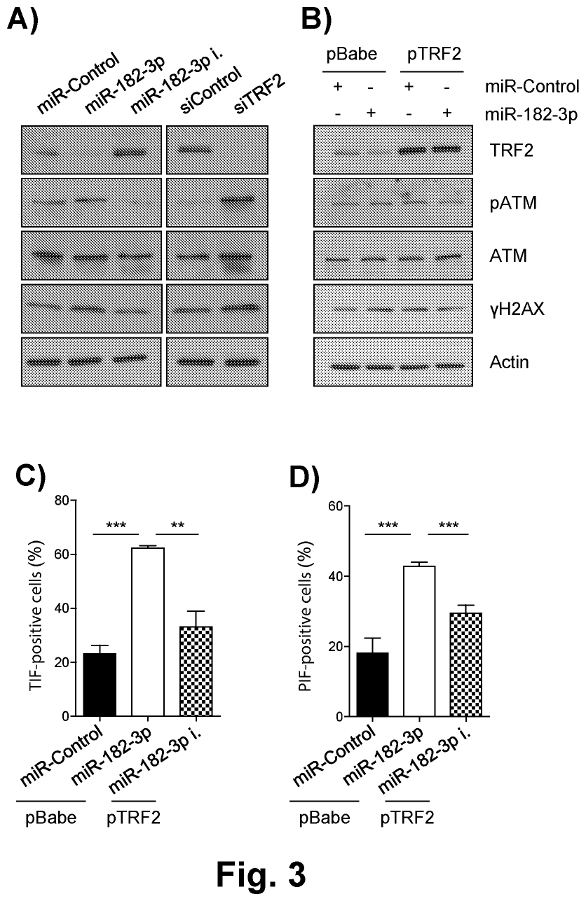

FIG. 3 shows that the ectopic expression of miR-182-3p induces damage to telomeric and pericentromeric DNA. Western blot of markers of DNA damage under normal conditions (A) and conditions of TRF2 overexpression (B). Results of immunofluorescence combined with FISH DNA, which show induction of the formation of foci of telomeric (C) and pericentromeric (D) DNA damage. Experiments conducted on the MDA-MB-231 line (TNBC).

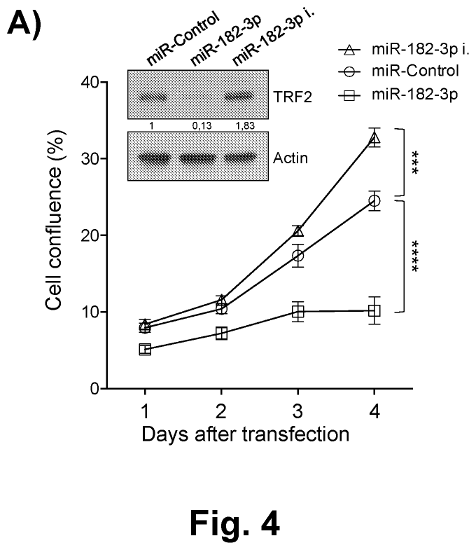

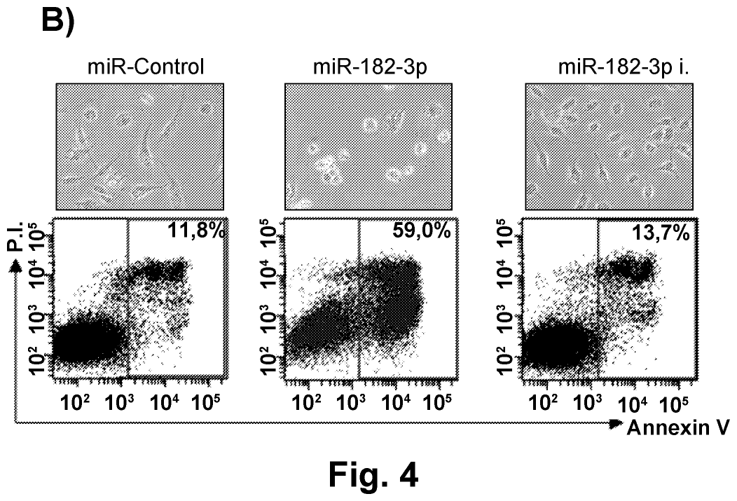

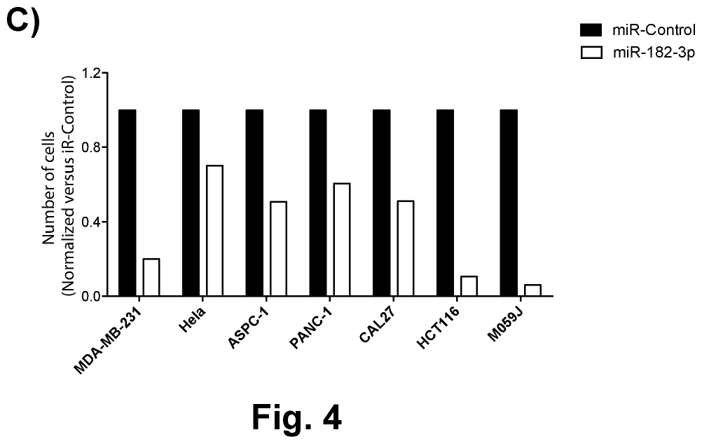

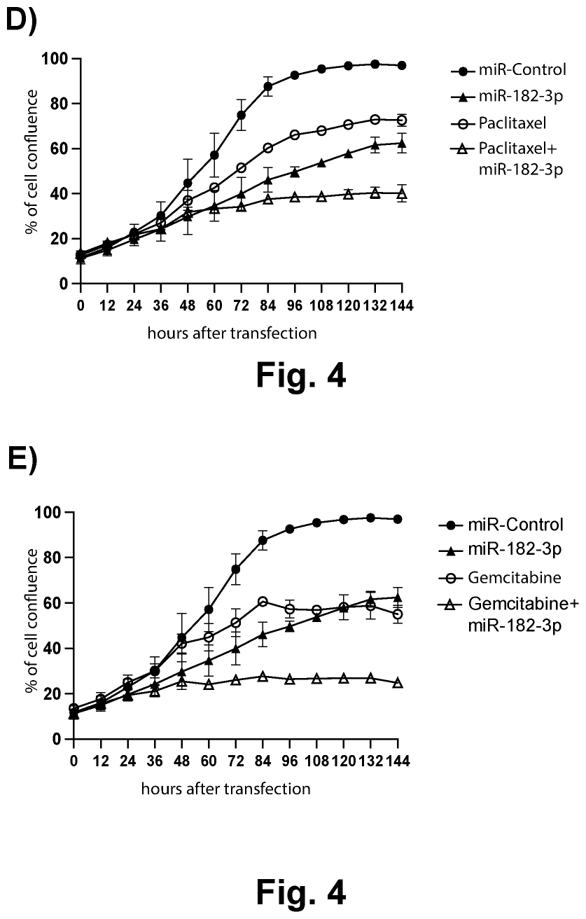

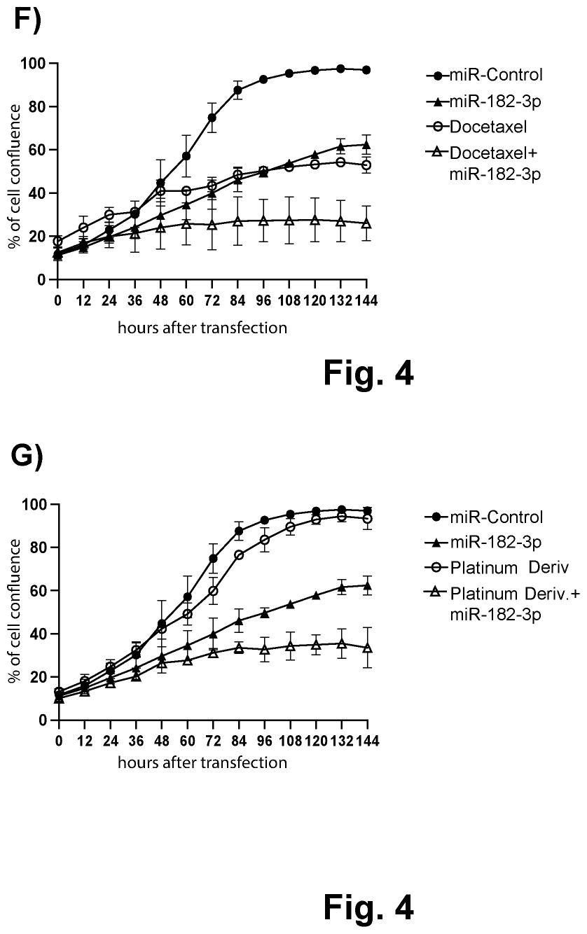

FIG. 4 shows that the ectopic expression of miR-182-3p on its own or in combination with drugs for clinical use, inhibits cell proliferation and induces apoptosis. A. Data on cell proliferation acquired by means of the IncuCyte system. Western blot on TRF2 following ectopic expression of miR-182-3p and the inhibitor thereof. B. Results of annexin V marking which show the induction of apoptosis. Representative images acquired immediately before the cells were collected. Experiments conducted on the MDA-MB-436 (TNBC) line. C The bar graph shows the number of cells after two cycles of transfection with the control miR (miR CTR) or miR 182-3p. The tumour lines used are: MDA-MB-231 (triple negative breast cancer), HeLa (cervical cancer), HCT116 (colon cancer), ASPC-1 and PANC-1 (pancreatic cancer), CAL-27 (mouth cancer), M059J (glioblastoma). D-G cell proliferation data acquired by means of the IncuCyte system in MDA-MB-231 cells transfected with the control miR (miR CTR) or miR 182-3p and treated with the drugs Paclitaxel (D), Gemcitabine (E), Docetaxel (F) and a platinum derivative (G).

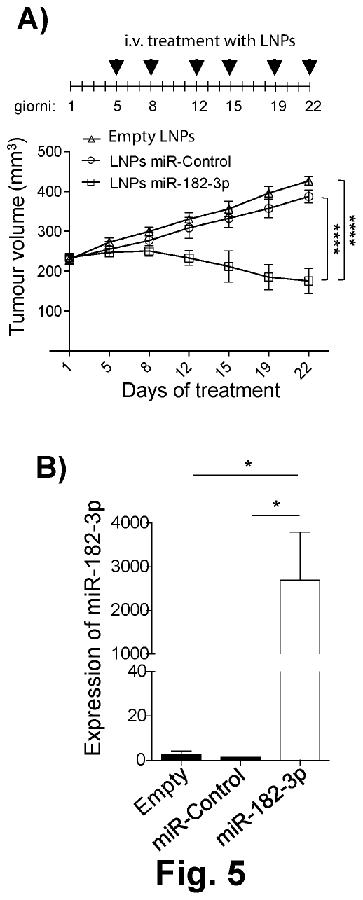

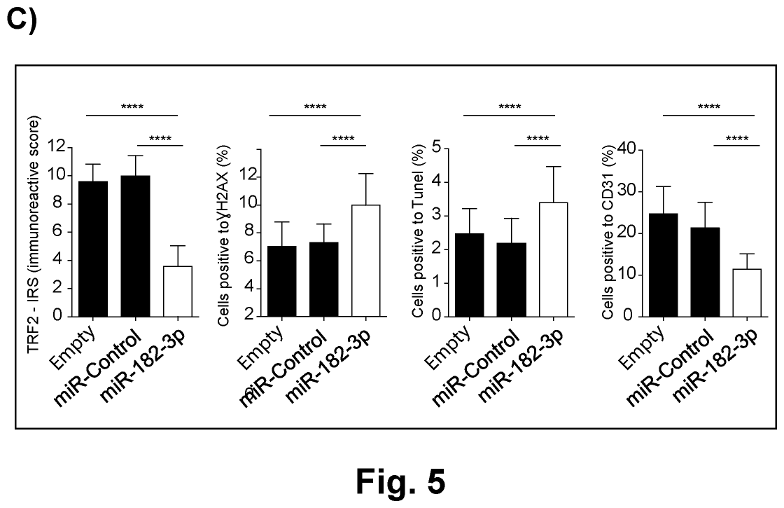

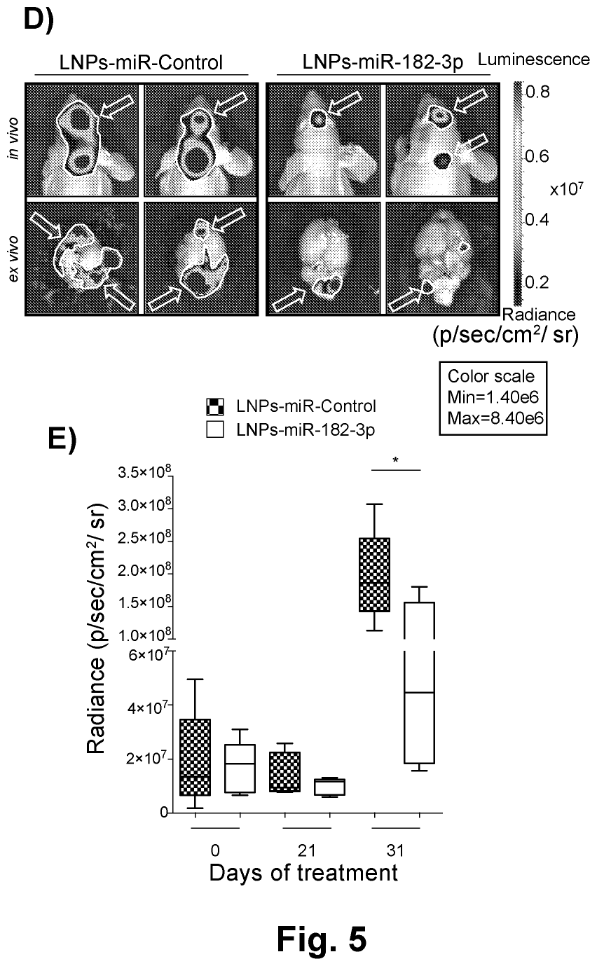

FIG. 5 shows that LNPs-miR-182-3p reduces tumour growth in vivo by modulating TRF2 in TNBC models. A. Antitumour activity of miR-182-3p encapsulated in lipid nanoparticles (LNPs) in an intramuscular implant model. B. Analysis of miR-182-3p expression in the treated tumours. C. Results of the immunohistochemical analysis on TRF2, γH2AX, CD31 and TUNEL staining in the treated tumours. D. Representative images of the antitumour effectiveness of treatment with LNPs-miR-182-3p in the intracranial implant. The tumours detected by optical imaging are circled and indicated by arrows. E. Box plot representative of the quantification of the bioluminescence signal emitted by intracranial tumours. Experiments conducted by inoculating MDA-MB-436.

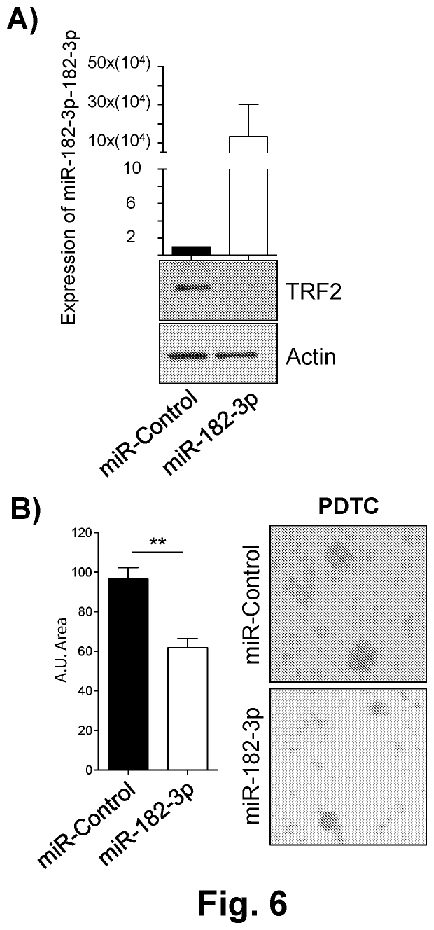

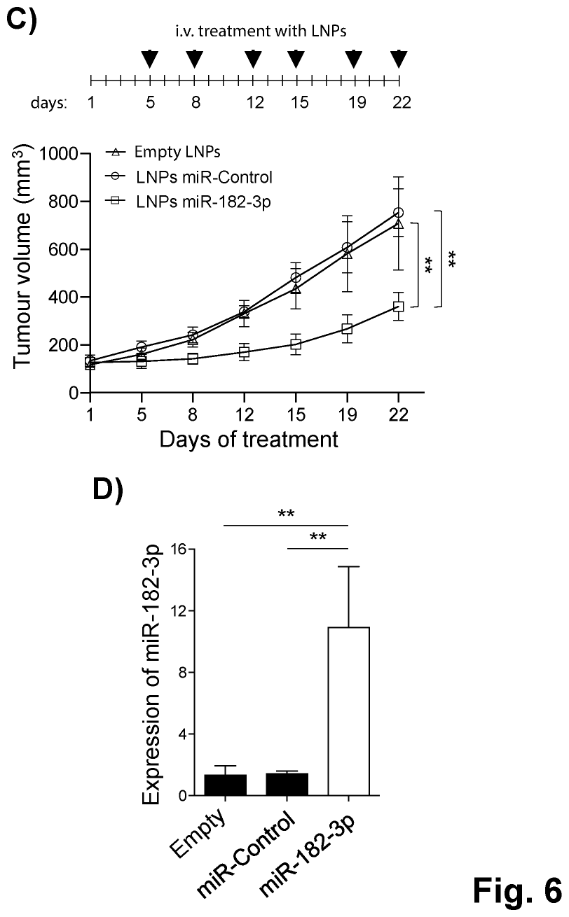

FIG. 6 shows that miR-182-3p reduces tumour growth in advanced PDTC and PDX models of triple negative breast cancer. A. Analysis of miR-182-3p expression and reduction of TRF2 in the PDTC culture and related effects on cell growth (B). C. Antitumour activity of miR-182-3p encapsulated in lipid nanoparticles (LNPs) in the PDX implant model and related intratumour absorption analysis (D).

### EXAMPLE 1. STUDY OF THE EFFECTIVENESS OF MIRNAS AS REGULATORS OF TRF2 EXPRESSION IN TUMOURS, IN PARTICULAR IN THE TREATMENT OF TRIPLE NEGATIVE BREAST CANCER (TNBC)

### Materials and Methods

Immunohistochemical Analysis on Samples from Patients

An immunohistochemical analysis of TRF2 was carried out on a series of 30 samples of primary triple negative breast cancer and the corresponding metastases, 55 samples of invasive breast cancer of different molecular subtypes (10 Luminal A, 10 Luminal B/HER2 negative, 10 Luminal B/HER2 positive, 9 HER2, 16 triple negative) and 50 samples of benign breast lesions with adjacent normal tissue (which may considered as 41 cases). All samples were surgically treated at the Regina Elena National Cancer Institute (Rome, Italia) between 2001 and 2018. The samples, fixed in formalin and embedded in paraffin, were cut on SuperFrost Plus slides (Menzel-Gläser, Braunschweig, Germany). The sections, 3 μm thick, were stained with Bond Polymer Refine Detection on an automated staining system (Bond™ Max, Leica Biosystems, Milan, Italy) with the anti-TRF2 antibody (4A794, Millipore). Diaminobenzidine (DAB) was used for the chromogenic substrate. Two biologists assessed the samples and those in which the immunostaining of TRF2 showed to be present in at least 10% of the neoplastic cells analysed were considered positive. The immunoreactive score (IRS) was calculated by multiplying the score of the positive cellular proportion by the score of the staining intensity, as described in Fedchenko et al.

### TCGA Dataset and Bioinformatic Analysis

The normalized TCGA-BRCA gene expression in the tumour samples was obtained from the Broad Institute TCGA Genome Data Analysis Center (http//gdac.broadinstitute.org/): Firehose stddata_2016_01_28. Broad Institute of MIT and Harvard. doi: 10.7908/C11G0KM9. The statistical significance of gene modulation among the different subgroups of samples was calculated by means of the Wilcoxon test. ANOVA was used to compare more than two groups. Significance was defined as p<0.05. Overall survival (OS) was assessed using Kaplan-Meier analysis and statistical significance was calculated with the log-rank test for the purpose of evaluating the difference between curves. Patients with a high and low signal intensity were defined based on the positive and negative z-scores, respectively. The analyses were completely conducted with Matlab R2020b.

### Cell Lines, Culture Conditions and Transfection

HeLa (human cervical cancer), HCT116 (colon cancer), U2-OS (osteosarcoma), Panc1 and AsPC-1 (pancreatic cancer), M059J (Glioblastoma), CAL27 (mouth cancer), MDA-MB-231 and MDA-MB-436 (human triple negative breast cancer) cell lines were purchased from ATCC. The HeLa, HCT116, U2-OS, M059J, MDA-MB-231 and MDA-MB-436 cell lines were cultured in Dulbecco's Modified Eagle's Medium (DMEM; Invitrogen, Carlsbad, CA, USA) with a high glucose content. The Panc1, AsPC-1, CAL27 cell lines were cultured in Roswell Park Memorial Institute 1640 Medium (RPMI; Gibco). Both media were supplemented with 10% foetal bovine serum (FBS, Hyclone), 1% penicillin-streptomycin and 1% L-glutamine at 37° C. with 5% CO2. The MDA-MB-436 cells were rendered luminescent by infection with the lentiviral vector pRRLSIN.cPPT.Luciferase (Addgene). The MDA-MB-231 consistently overexpressing TRF2 (pBabe-puro-mycTRF2) and the control counterpart (pBabe-puro-Empty) (Okamoto et al. 2013) were obtained by infecting the cells with amphotropic retroviruses generated in Phoenix cells transfected with retroviral vectors by means of JetPEI (Polyplus, New York, NY, USA).

For the RNA transfection experiments, use was made of MIR 182-3p, miR 182-3p-inhibitor and miR-Control (Ambion); siRNA siTRF2 (Dharmacon Inc., Chicago, USA) and siControl (Santa Cruz Biotechnology; CA, USA). The agent INTERFERin (Polyplus) was used for the transfections into cells. The drugs Paclitaxel (4 nM), Gemcitabine (10 nM), Docetaxel (10 nM), and platinum derivatives (8 μM) were used for the treatments. The treatment took place 24 h after transfection with miR-Control or miR-182-3p.

### Screening and Luciferase Assay

A computational analysis was carried out to identify the miRNAs specific for the 3′UTR of TRF2 using 3 specific software applications (PITA, TargetScan and micro-RNA.org) (Dinami et al. 2017). For the luciferase assays, the 3′-UTR of TRF2 was cloned downstream of the Renilla luciferase cassette in the psiCHECK2 vector (Promega). The Firefly reporter gene was used as an internal control of the expression of luciferase with respect to Renilla in order to evaluate transfection efficiency. High-throughput screening was conducted in Hela cells co-transfected with the 3′-UTR-TRF2 (18 ng) luciferase reporter plasmid and the candidate miRNAs (final concentration: 50 nM). Validation of the screening was then carried out by co-transfecting the mimic-miR-182-3p (10 nM, Ambion) with the wild-type 3′-UTR-TRF2 or the mutant of 3′-UTR-TRF2, wherein a deletion was present in the binding site for miR-182-3p (Q5 site-directed Mutagenesis Kit, NEB). 72 hours after transfection, the activity of the Renilla/Firefly luciferase reporter was analysed using a Dual Luciferase Reporter Assay System (Promega) and a GloMax 96 (Promega) microplate luminometer. Experiments conducted at least in duplicate. Statistical analysis conducted by means of a t-test.

### Western Blot

The cells were collected and lysed as previously described (lachettini et al. 2018). The TRF2 expression levels were evaluated using the anti-TRF2 monoclonal antibody (Millipore, 4A794). The response to DNA damage was evaluated using the following antibodies: p-ATM mAb (Ser1981), anti-ATM mAb, (Cell Signaling Technology, Beverly, MA, USA); and anti-γH2AX mAb (Ser139) (Millipore, Bedford, MA). Actin, detected with the mouse monoclonal anti-β-actin antibody (Sigma Aldrich), was used as a loading control. The protein band intensity was quantified by densitometric analysis using ImageJ software (http://rsb.info.nih.gov/ij/). Experiments conducted in triplicate. Statistical significance calculated by means of a t-test.

Immunofluorescence Combined with DNA-FISH

The cells were fixed and subjected to fluorescence in situ hybridization as previously described (Salvati et al. 2015). The analysis of fluorescence was performed by confocal laser scanning microscopy using a Zeiss LSM 880 with Airyscan (Zeiss, Germany). For TIF quantification, nuclei with at least three colocalizations between γH2AX (anti-phospho H2A.X mAb, Millipore) and the telomere probe (TelC-Cy3 PNA probe, Panagene) were considered positive. For PIF quantification, by contrast, nuclei with at least one colocalization between the pericentromeric probe (Cy3-labeled Satill PNA probe, Panagene) and γH2AX were considered positive. Statistical analysis conducted by means of a t-test.

### RNA Extraction and TaqMan Assay

The total RNA was isolated from the cell lines using the TRIzol reagent (Invitrogen, Carlsbad, CA, USA). The quality and quantity of RNA extraction were evaluated based on the A260 nm/A280 nm and A260 nm/A230 nm absorbance ratio (Nanodrop 1000, ThermoFisher Scientific). The analysis of miRNA expression was conducted by means of the TaqMan™ Universal Master Mix II, no UNG (Applied Biosystems) using the specific probes (Applied Biosystems). The TaqMan assay was performed with the QuantStudio 6 Flex Detection system (ThermoFisher). Experiments performed in triplicate. Statistical analysis conducted by means of a t-test.

### Flow Cytometry

An analysis of the cell cycle was performed by flow cytometry (Becton-Dickinson) after cell staining with propidium iodide (PI), as previously described (Zizza et al. 2019). The cells were analysed with a FACSCelesta (BD Biosciences, San Jose, CA, USA). Cell progression throughout the phases of the cell cycle was analysed by means of simultaneous flow cytometry measurements of the DNA and 5-bromo-2′-deoxyuridine (BrdU) content of the cells, as previously described (Biroccio et al. 2001).

Apoptosis was evaluated by labelling with annexin V and propidium iodide (PI), as previously described (Biroccio et al. 2002). The assay was conducted using FACSCelesta and the data were analysed using FACS Diva Software (BD Biosciences, San Jose, CA, USA).

### Cell Proliferation

The cells were transfected with the miR-Control, miR-182-3p or 182-3p-inhibitor (10 nM). After 72 hours, the cells were collected, counted and reseeded in order to carry out the second transfection cycle. Cell growth was monitored with the Incucyte® S3 system (Essen BioScience, Ann Arbor, MI) by acquiring images every 12 or 24 hours. Viability was evaluated by comparing cell confluence among the groups using IncuCyte S3 software (Essen BioScience). Experiments performed in triplicate. Statistical significance calculated by means of a t-test.

The cell count was performed by means of a Countess semi-automated counter (ThermoFisher Scientific).

### PDTC

In order to obtain the PDTC culture, a fragment of a patient-derived tumour xenograft (PDX) was subjected to mechanical and enzymatic dissociation following the soft tissue tumour dissociation protocol on a GentleMACS Dissociator and a human tumour dissociation kit (Miltenyi Biotec, Cat ID 130-093-235) according to the manufacturer's instructions. After a homogeneous suspension of single cells was obtained, they were plated at a density of 2×10⁵ and subjected to two transfection cycles. The inhibition of growth was calculated by calculating the area occupied by cells on the plate using ImageJ software. Experiments performed in triplicate. Statistical analysis performed by means of a t-test.

### Preparation of Lipid Nanoparticles

The formulations of the lipid nanoparticles (LNPs), empty or containing the miRNAs, were prepared by means of the ethanol injection method, as previously described (Fattore et al. 2020). The DSPC/CHOL/DODAP/PEG2000-Cer16 (25/45/20/10 w/w) lipid solution was prepared in ethanol (40% v/v). A 0.2 mg aliquot of miRNA-Control or miR-182-3p was dissolved in 20 mM of citric acid at pH 4.0. The two solutions were heated to a temperature of 65° C. and the lipid solution was subsequently added drop by drop to the respective solutions of miRNAs under stirring. The preparation was measured by means of 200 and 100 nm polycarbonate filters using a Thermobarrel extruder (Northern Lipids Inc., Vancouver, BC, Canada) at 65° C. The preparation was then dialyzed (cutoff 3.5 kDa) in a citrate buffer (20 mM, pH 4.0) for about an hour to remove the excess ethanol and in HBS (20 mM HEPES, 145 mM NaCl, pH 7.4) for 12-18 hours to remove the citrate buffer and neutralise the surface of the LNPs. The quantity of non-encapsulated miRNAs was removed by ultracentrifugation at 278,835 g for 40 minutes (Optima Max E, Beckman Coulter, USA; rotor TLA 120.2).

For the characterization of the nanoparticles, the particle size, particle size distribution (PI) and z potential (ZP) were measured by dynamic light scattering with a Zetasizer Ultra (Malvern Instruments, Worcestershire, United Kingdom). The samples were diluted (1:100 v/v) with water filtered at 0.22 μm and analysed. The results were obtained by averaging the measurements over three different batches of the same formulation. In order to measure the quantity of encapsulated miRNA, the formulations were dissolved in methanol (1:100 v/v) and the samples were centrifuged at 16,250 g for 30 minutes (MIKRO 20; Hettich, Tuttlingen, Germany). The supernatants were then analysed by spectrophotometry at 260 nm. The encapsulation efficiency (EE %) was calculated as the % ratio between the actual miRNA load and the theoretical miRNA load in the formulation (mg of miRNAs/mg of total lipids). The results, calculated by averaging the measurements over three different batches, are reported in Table 1. In particular, Table 1 shows the characterization of the lipid nanoparticles (LNPs) in terms of size, polydispersity index (PI), z potential (ZP), actual load (μg miRNAs/mg lipids) and encapsulation efficiency (EE %).

TABLE 1

| | | | | | Actual | |
|---|---|---|---|---|---|---|
| | | Diameter | PI ± | ZP | loading ± | EE % ± |
| Formulation | miRNA | nm ± SD | SD | mV ± SD | SD | SD |
| Empty | — | 121.7 ± 15.6 | 0.12 ± 0.06 | −8.5 ± 4.8 | — | — |
| LNPs | | | | | | |
| LNP-miR- | miR- | 140.0 ± 11.3 | 0.17 ± 0.04 | −14.7 ± 6.2 | 192.4 ± 4.1 | 96 ± 2.1 |
| Control | Control | | | | | |
| LNP-miR- | miR-182- | 152.9 ± 15.3 | 0.20 ± 0.04 | −18.5 ± 5.6 | 194.1 ± 1.8 | 97 ± 0.8 |
| 182-3p | 3p | | | | | |

DODAP (1,2-dioleoyl-3-dimethylammonium-propane) and PEG2000-Cer16 (N-palmitoyl-sphingosine-1-{succinyl [methoxy (polyethylene glycol) 2000]}) were purchased from Avanti Polar Lipids. DSPC (Distearoylphosphatidylcholine) was kindly offered by Lipoid GmbH (Cam, Switzerland). Cholesterol (CHOL), sodium chloride, sodium phosphate, HEPES, citric acid and sodium citrate were purchased from Sigma Aldrich (USA). Ethanol and other solvents were obtained from Exacta Optech (Italy).

### Animal Models

All procedures on animals complied with national and international directives (Decree Law no. 26 of 4 Mar. 2014, n. 26; Directive 2010/63/EU of the European Parliament and of the Council; Guide for the Care and Use of Laboratory Animals, United States National Research Council, 2011; Animal Research Reporting of In Vivo Experiments (ARRIVE) guidelines) and were approved by the Italian Ministry of Health (authorisation no. 607/2019-PR, issued on Jul. 8, 2019).

Female CB17-SCID mice (CB17/lcr-Prkdcscid/IcrlcoCrl, #236. Charles River Laboratories, Calco, Italy) were inoculated intramuscularly with 4×106 MDA-MB-436 cells. When the tumour volume had reached about 250 mm³, the animals were randomized into three different groups and treated. Tumour growth was monitored by using a calliper and calculating the tumour volume by following the formula (a²×b)/2, where a and b are respectively the smallest and largest measurement of the mass. Female nude mice (Athymic Nude-Foxn1^{nu}, 069-IT. Envigo, San Pietro al Natisone (UD), Italy) were inoculated intracranially with 1.8×10⁵ luminescent MDA-MB-436 cells. The treatments began one week after following the previously adopted administration regime. Tumour growth was monitored by using IVIS optical imaging (PerkinElmer). Female NSG mice (NOD.Cg-PrkdcSCID IL-2R null, #614. Charles River Laboratories, Calco, Italy) were inoculated subcutaneously with a fragment of about 25-30 mm³ of directly patient-derived triple negative breast cancer. The tumours were expanded in a growing number of animals in order to reach a suitable quantity for the experiment. When the tumour volume had reached about 150 mm³, the mice were randomized into three groups and treated. Tumour growth was monitored by calibration.

The LNPs were administered intravenously five times (20 μg/die), with a three-day interval between injections for a total of six treatments. Statistical analyses conducted by means of t-tests.

Immunohistochemical Analysis of Samples from Mice

Tissue samples collected from tumours and main organs were fixed in formalin, embedded in paraffin and sectioned (2 μm) for haematoxylin and eosin staining. For immunostaining, the sections were deparaffinated, rehydrated and subjected to antigen retrieval by means of PT Link (Dako Omnis) with blocking of peroxidase and nonspecific bonds (Dako Omnis solutions). Immunostaining of the sections was carried out with the following primary antibodies: anti-TRF2 (Novus Biologicals, 4A794.15, 1:500, mouse), anti-γH2AX (Bethyl Laboratories, BLR059F, 1:500, rabbit), and anti-CD31 (Dianova, SZ31, 1:10, rat). The sections were incubated for 30 minutes with Dako EnVision™ FLEX/HRP (EnVision™ FLEX Dako Omnis). Detection of apoptotic cells by means of the TUNEL test-mediated by terminal deoxy-transferase—was carried out using the In Situ Cell Death Detection Kit-POD (Roche Molecular Biochemicals). The signal was developed using DAB (EnVision™ FLEX Dako Omnis). Images were acquired with the Aperio ScanScope CS System and the results were evaluated as a percentage of positive cells or an immunoreactive score (IRS) (Fedchenko & Reifenrath, 2014).

### Results

TRF2 Expression Positively Correlates with the Progression of Breast Cancer, Metastasis Formation and a Worse Prognosis for Patients

With the aim of exploring the clinical relevance of TRF2 in breast tumour formation and progression, TRF2 levels were analysed in human samples of normal breast tissue, benign lesions and malignant tumours surgically treated at the Regina Elena National Cancer Institute. It was observed that TRF2 expression progressively increases from normal tissue to benign lesions and becomes even higher in malignant tumours (FIG. 1A, B). In order to establish whether TRF2 expression differs among the various subtypes of malignant tumours, use was made of a broader cohort of patients with breast cancer drawn from the TCGA dataset (The Cancer Genome Atlas). TRF2 mRNA expression increases progressively with the growing aggressiveness of the tumour subtypes, reaching the highest level in the basal subtype (FIG. 1C). Moreover, TRF2 expression is greater in oestrogen and progesterone receptor-negative and HER2-negative breast cancers (triple negative), which are characterised by a more aggressive clinical course, early recurrence and a poor prognosis (FIG. 1D).

TRF2 expression in primary triple negative tumours and the corresponding metastatic lesions thereof were subsequently analysed in a cohort of patients surgically treated at the Regina Elena National Cancer Institute. A significant increase in TRF2 expression was found in metastatic lesions compared to primary tumours irrespective of the site of metastasis (local recurrence or various distant organs) and irrespective of the route of spread (blood or lymphatic system) (FIG. 1E, F). These results indicate that TRF2 plays a fundamental role in promoting the metastatic process in human patients with TNBC.

Finally, when the breast cancer patients (TCGA dataset) were stratified on the basis of TRF2 mRNA expression (FIG. 1G), it was found that the patients with the highest TRF2 levels showed a worse clinical outcome.

Overall, these data identify TRF2 as a new marker of breast cancer progression and prognosis.

Screening by Means of Luciferase Assay Enabled the Identification of miR-182-3p, miR-519e-5p and miR-296-3p as New Regulators of TRF2 Expression

With the aim of developing a strategy based on the use of miRNAs to combat TRF2 in human cancer, a multi-phase approach was adopted. Starting from a comparison of the computational analyses of three different software applications, a list of 54 candidate miRNAs was extrapolated, based on their specificity for the 3′-UTR of TRF2. In order to validate these results, the effectiveness of the miRNAs was tested through high-throughput screening based on a luciferase assay (FIG. 2A). Briefly, Hela cells were transfected in a transient manner both with a reporter vector containing luciferase cDNA fused to the 3′-UTR of TRF2 and each of the synthetic miRNAs (FIG. 2A). By measuring the luciferase activity three days after transfection, it was possible to identify three miRNAs (miR-182-3p, miR-519e-5p, miR-296-3p) capable of reducing the luminescence signal by about 50% (FIG. 2A). Subsequently, these three miRNAs were individually transfected and their ability to reduce the TRF2 protein levels was evaluated. All three of the tested miRNAs were capable of significantly reducing the TRF2 protein levels. In particular, miR-519e-5p induced a reduction of about 30% in TRF2 expression, miR-296-3p a reduction of about 50%, whilst miR-182-3p proved to be the most efficient, given its ability to reduce the expression of TRF2 by 75% (FIG. 2B).

These data identified the miRNAs miR-182-3p, miR-296-3p and miR-519e-5p as new regulators of TRF2 expression.

Subsequently, in order to confirm the specificity of miR-182-3p, a reporter construct was generated in which the 3′-UTR of TRF2 had undergone a deletion at the target site of miR-182-3p (mut). The reduction in luciferase activity was confirmed in the assay with the wild type construct, whereas no significant effect was observed with the deleted construct (FIG. 2C, D). Subsequently, in order to verify that the activity of the miRNAs was not dependent on the cellular context, miR-182-3p was ectopically expressed in different tumour cell lines (Hela, HCT116, MDA-MB-231, MDA-MB-436 and U2-OS). It was thus observed that the overexpression of miR-182-3p markedly reduced the TRF2 protein levels in all tested models of tumour cells (FIG. 2E).

These data identified miR-182-3p as a new specific regulator of TRF2 expression. Accordingly, the effects of this miRNA were studied in several tumour models, including triple negative breast cancer.

### MIR-182-3p Induces Damage to Telomeric and Pericentromeric DNA in TNBC Cells

Recent evidence has shown an extratelomeric role of TRF2 in maintaining genome stability, demonstrating that the protein knockdown triggers the activation of DNA damage also in the pericentromeric regions (Mendez-Bermudez A. et al. 2018). Therefore, the effects of the ectopic expression of miR-182-3p in the MDA-MB-231 (TNBC) cell line were studied by analysing the markers of the DNA damage response. The results showed that the transient transfection of miRNA, similarly to the knockdown mediated by a specific siRNA, induced an increase in the levels of pATM and γH2AX (FIG. 3A). It is worth noting that suppressing the endogenous miR-182-3p using a specific inhibitor (miR-182-3p i.) increased the TRF2 levels, with a consequent reduction of pATM and γH2AX (FIG. 3A). Therefore, for the purpose of confirming that the DNA damage was localised to telomeres and pericentromeres, the formation of telomere dysfunction-induced foci (TIFs) and pericentromere dysfunction-induced foci (PIFs) was evaluated by immunofluorescence analysis of γH2AX combined with DNA FISH, using, respectively, the specific Tel-C and Satill probes. An increase in damage both in the telomeric regions and pericentromeric regions was observed from the data as a result of ectopic expression of miR-182-3p (FIG. 3C, D). Moreover, it was interesting to note that the increase in DNA damage was in general countered to a significant decree by the overexpression of TRF2 (pTRF2), demonstrating that the modulation of TRF2 was the cause for the activation of DNA damage (FIG. 3B-D).

Overall, these results indicate that, by regulating the expression of TRF2, miR-182-3p triggers a general activation of DNA damage in triple negative breast cancer cells, with a specific impact on the telomeric and pericentromeric regions.

Ectopic Expression of miR-182-3p Reduces Cell Proliferation in Various Tumour Lines and Induces Apoptosis in TNBC Cells

In the light of the results obtained regarding induced DNA damage, it was desired to verify whether miR-182-3p could rapidly promote the inhibition of growth and/or death of tumour cells. Therefore, the effects of the ectopic expression of miR-182-3p on cell proliferation were verified in the MDA-MB-436 (TNBC) line. Using the IncuCyte instrument, the growth of cells transfected with miR-182-3p, miR-182-3p I, and the negative control (miR-Control) was measured in the time-lapse mode. The analysis of the acquired images showed that the overexpression of miR-182-3p inhibited cell growth by over 50% at the second transfection cycle (FIG. 4A), an effect correlated with the downregulation of TRF2 and the change in cell morphology (FIG. 4A, B). In contrast, transfection with the miR-182-3p inhibitor increased cell confluence compared to the control (FIG. 4A).

The analysis of annexin V subsequently confirmed that miR-182-3p greatly increased the percentage of cells undergoing apoptosis (FIG. 4B). The treatment with the miR-182-3p inhibitor, by contrast, notwithstanding the result in the proliferation assay, did not induce a reduction in apoptotic cells compared to the control (FIG. 4B). Moreover, based on the known overexpression of TRF2 in various types of tumours (Cherfils-Vicini et al. 2019, Figure EV5), the miR-182-3p-mediated effects on proliferation were also validated in other tumour cell lines (MDA-MB-231, HeLa, HCT116, M059J, PANC-1, AsPC-1, CAL27) by means of a cell count (FIG. 4C). Finally, it was observed that a synergy exists between miR-182-3p and drugs for clinical use such as Paclitaxel, Gemcitabine, Docetaxel and platinum derivatives. In fact, the combination of miR-182-3p with the various tested drugs shows a greater antiproliferative effect compared to treatment with miR-182-3p alone (FIG. 4D-G).

These data demonstrate that miR-182-3p exerts an antiproliferative effect on various types of tumour cells, in particular on triple negative breast cancer cells, by triggering apoptosis. Moreover, the combined treatment of miR-182-3p with drugs for clinical use enhances the antiproliferative effect.

### MIR-182-3p Encapsulated in Lipid Nanoparticles Inhibits Tumour Growth In Vivo in TNBC Models

With the aim of further investigating the effects of treatment with miR-182-3p, the in vivo study was continued by encapsulating the double-stranded miRNA in lipid nanoparticles (LNPs). The first experimental model used was intramuscular inoculation of the MDA-MB-436 line in immunodeficient mice. The animals were treated intravenously with empty nanoparticles (LNPs-Empty) as a negative control, nanoparticles with a targetless control miRNA (LNPs-miR-Control), or nanoparticles with miR-182-3p (LNPs-miR-182-3p). Whereas the control miRNA did not show any antitumour activity, treatment with LNPs-miR-182-3p exhibited broad effectiveness, even going so far as to induce tumour regression (FIG. 5A). At the end of the treatments, the mice were sacrificed for the ex-vivo analyses. The toxicity study ruled out systemic damage, simultaneously with the results of the TaqMan assay, which revealed an overexpression of miR-182-3p both in the tumours (FIG. 5B) and in organs, including the brain (data not shown). The immunohistochemical analyses later showed a significant intratumour reduction in TRF2 in the samples treated with LNPs-miR-182-3p versus the controls (FIG. 5C) and an increase in DNA damage and in the activation of apoptosis, as indicated by the significant increase in cells positive both for γH2AX and for staining with the TUNEL assay (FIG. 5C). Moreover, consistently with the previously demonstrated pro-angiogenic role of TRF2 (Zizza et al. 2019), a lower formation of vessels was observed in the tumours treated with LNPs-miR-182-3p, as demonstrated by the reduction in the specific marker CD31 (FIG. 5C).

Finally, the treatment was tested on brain metastases artificially generated by directly implanting luminescent MDA-MB-436 cells in the animals' brains, a preclinical metastatic model that mimics one of the most dramatic stages of cancer progression that occurs in patients with triple negative breast cancer (Soto and Sibson, 2016). The quantitative analysis of bioluminescence revealed a reduction in the size of the brain tumour mass in mice treated with LNPs-miR-182-3p (FIG. 5D, E)

These results strongly suggest that the inhibition of tumour growth caused by treatment with LNPs-miR-182-3p is specifically guided by the repression of TRF2. Furthermore, these results indicate an optimal absorption of nanoparticles, which are also capable of crossing the blood-brain barrier.

### MIR-182-3p Induces the Inhibition of Tumour Growth in Advanced Preclinical Models of TNBC

Human tumours show inter- and intratumour heterogeneity which decidedly influences the response to antitumour therapies (Aparicio and Caldas, 2013). To date, patient-derived xenografts (PDX) of tumours and cultures of cells derived therefrom (PDX-derived tumour cells, or PDTC) represent the best preclinical models for screening antitumour drugs, precisely because of their characteristics of maintaining the clonal and molecular heterogeneity of the original tumour (Bruna et al. 2016). Therefore, the therapeutic response to treatment with miR-182-3p was evaluated in both models. For this purpose, PDTC cultures were first generated from a triple negative breast cancer, mutated in BRCA1 (germline mutation) and resistant to PARP inhibitors (Olaparib), thus particularly aggressive. The cultures were subjected to a double cycle of transfection with miR-182-3p and the control miRNA. The ectopic expression of miR-182-3p, verified by means of the TaqMan assay (FIG. 6A), reduced the expression of TRF2 and inhibited cell growth (FIG. 6A, B). Subsequently, the effectiveness of the treatment with LNPs-miR-182-3p was tested in the corresponding PDX model. More specifically, when the tumours implanted subcutaneously in NSG female mice reached a volume of about 150 mm³, the animals were divided into three groups and treated with LNPs-Empty,-miR-Control or miR-182-3p. At the end of the treatments, it was possible to observe a 50% reduction in the tumour volume in mice treated with LNPs-miR-182-3p, parallel to a significant intratumour miRNA absorption (FIG. 6C, D).

These data confirm the tumour-suppressive activity of miR-182-3p also in highly aggressive heterogeneous preclinical models.

### REFERENCES

Aparicio S, Caldas C. The implications of clonal genome evolution for cancer medicine. N Engl J Med. 2013 Feb. 28; 368 (9): 842-51.
Bardia A, Mayer I A, Vahdat L T, Tolaney S M, Isakoff S J, Diamond J R, O'Shaughnessy J, Moroose R L, Santin A D, Abramson V G, Shah N C, Rugo H S, Goldenberg D M, Sweidan A M, Iannone R, Washkowitz S, Sharkey R M, Wegener W A, Kalinsky K. Sacituzumab Govitecan-hziy in Refractory Metastatic Triple-Negative Breast Cancer. N Engl J Med. 2019 Feb. 21; 380 (8): 741-751.
Benhamou Y, Picco V, Raybaud H, Sudaka A, Chamorey E, Brolih S, Monteverde M, Merlano M, Nigro Lo C, Ambrosetti D et al. Telomeric repeat-binding factor 2: a marker for survival and anti-EGFR efficacy in mouth cancer. Oncotarget. 2016 Jul. 12; 7 (28): 44236-44251.
Biroccio A, Benassi B, D'Agnano I, D'Angelo C, Buglioni S, Mottolese M, Ricciotti A, Citro G, Cosimelli M, Ramsay R G, Calabretta B, Zupi G. c-Myb and Bcl-x overexpression predicts poor prognosis in colorectal cancer: clinical and experimental findings. Am J Pathol. 2001 April 158 (4): 1289-99.
Biroccio A, Benassi B, Filomeni G, Amodei S, Marchini S, Chiorino G, Rotilio G, Zupi G and Ciriolo M. Glutathione influences c-Myc-induced apoptosis in M14 human melanoma cells. J Biol Chem. 2002 November 277, 43763-43770.
Biroccio A, Cherfils Vicini J, Augereau A, Pinte S, Bauwens S, Ye J, Simonet T, Horard B, Jamet K, Cervera L et al. TRF2 inhibits a cell-extrinsic pathway through which natural killer cells eliminate cancer cells. Nat Cell Biol. 2013 July; 15 (7): 818-828.
Bruna A, Rueda O M, Greenwood W, Batra A S, Callari M, Batra R N, Pogrebniak K, Sandoval J, Cassidy J W, Tufegdzic-Vidakovic A, Sammut S J, Jones L, Provenzano E, Baird R, Eirew P, Hadfield J, Eldridge M, McLaren-Douglas A, Barthorpe A, Lightfoot H, O'Connor M J, Gray J, Cortes J, Baselga J, Marangoni E, Welm A L, Aparicio S, Serra V, Garnett M J, Caldas C. A Biobank of Breast Cancer Explants with Preserved Intra-tumour Heterogeneity to Screen Anticancer Compounds. Cell. 2016 Sep. 22; 167 (1): 260-274.e22.
Cherfils-Vicini J, Iltis C, Cervera L, Pisano S, Croce O, Sadouni N, Gyorffy B, Collet R, Renault V M, Rey-Millet M, Leonetti C, Zizza P, Allain F, Ghiringhelli F, Soubeiran N, Shkreli M, Vivier E, Biroccio A & Gilson E. Cancer cells induce immune escape via glycocalyx changes controlled by the telomeric protein TRF2. EMBO J. 2019 Jun. 3; 38 (11): e100012.
Di Martino M T, Campani V, Misso G, Gallo Cantafio M E, Gullà A, Foresta U, Hiram Guzzi P, Castellano M, Grimaldi A, Gigantino V, Franco R, Lusa S, Cannataro M, Tagliaferri P, De Rosa G, Tassone P, Caraglia M. In vivo activity of miR-34a mimics delivered by stable nucleic acid lipid particles (SNALPs) against multiple myeloma. PLOS One. 2014 Feb. 27; 9 (2): e90005.
Dinami R, Buemi V, Sestito R, Zappone A, Ciani Y, Mano M, Petti E, Sacconi A, Blandino G, Giacca M, Piazza S, Benetti R, Schoeftner S. Epigenetic silencing of miR-296 and miR-512 ensures hTERT dependent apoptosis protection and telomere maintenance in basal-type breast cancer cells. Oncotarget. 2017 Sep. 23; 8 (56): 95674-95691.
Fattore L, Campani V, Ruggiero C F, Salvati V, Liguoro D, Scotti L, Botti G, Ascierto P A, Mancini R, De Rosa G, Ciliberto G. In vitro biophysical and biological characterization of lipid nanoparticles Co encapsulating oncosuppressors miR-199b-5p and miR-204-5p as potentiators of target therapy in metastatic melanoma. International Journal of Molecular Sciences. 2020 Mar. 21 (6), 1930.
Fedchenko, N. & Reifenrath, J. Different approaches for interpretation and reporting of immunohistochemistry analysis results in the bone tissue—a review. Diagn. Pathol. 9, 221 (2014).
Iachettini S, Trisciuoglio D, Rotili D, Lucidi A, Salvati E, Zizza P, of Leo L, of the Bufalo D, Ciriolo M R, Leonetti C, Steegborn C, Mai A, Rizzo A, Biroccio A. Pharmacological activation of SIRT6 triggers lethal autophagy in human cancer cells. Cell Death Dis. 2018 Sep. 24; 9 (10): 996.
Lee S W L, Paoletti C, Campisi M, Osaki T, Adriani G, Kamm R D, Mattu C, Chiono V. MicroRNA delivery through nanoparticles. J Control Release. 2019 Nov. 10; 313:80-95.
Mendez-Bermudez A, Lototska L, Bauwens S, Giraud-Panis M J, Croce O, Jamet K, Irizar A, Mowinckel M, Koundrioukoff S, Nottet N, Almouzni G, Teulade-Fichou M P, Schertzer M, Perderiset M, Londoño-Vallejo A, Debatisse M, Eric Gilson E, Ye J. Genome-wide Control of Heterochromatin Replication by the Telomere Capping Protein TRF2. Mol Cell. 2018 May 3; 70 (3): 449-461.e5.
Müller R H, Mäder K, Gohla S. Solid lipid nanoparticles (SLN) for controlled drug delivery—a review of the state of the art. Eur J Pharm Biopharm. 2000 July 50 (1): 161-77.
Nakanishi K, Kawai T, Kumaki F, Hiroi S, Mukai M, Ikeda E, Koering C-E, Gilson E. Expression of mRNAs for telomeric repeat binding factor (TRF)-1 and TRF2 in atypical adenomatous hyperplasia and adenocarcinoma of the lung. Clin Cancer Res. 2003 March; 9 (3): 1105-1111.
O'Brien J, Hayder H, Zayed Y, Peng C. Overview of MicroRNA Biogenesis, Mechanisms of Actions, and Circulation. Front Endocrinol (Lausanne). 2018 Aug. 3; 9:402.
Okamoto, K.; Bartocci, C.; Ouzounov, I.; Diedrich, J. K.; Yates, J. R.; Denchi, E. L. A Two-Step Mechanism for TRF2-Mediated Chromosome-End Protection. Nature. 2013 494, 502-505.
Ozcan G, Ozpolat B, Coleman R L, Sood A K, Lopez-Berestein G. Preclinical and clinical development of siRNA-based therapeutics. Adv Drug Deliv Rev. 2015 Jun. 29; 87:108-19.
Rivel T, Ramseyer C, Yesylevskyy S. The asymmetry of plasma membranes and their cholesterol content influence the uptake of cisplatin. Sci Rep. 2019 Apr. 4; 9 (1): 5627.
Salvati E, Rizzo A, lachettini S, Zizza P, Cingolani C, D'Angelo C, Porru M, Mondello C, Aiello A, Farsetti A, Gilson E, Leonetti C, Biroccio A. A basal level of DNA damage and telomere deprotection increases the sensitivity of cancer cells to G-quadruplex interactive compounds. Nucleic Acids Res. 2015 Feb. 18; 43 (3): 1759-69.
Soto M S and Sibson N R. Mouse Models of Brain Metastasis for Unravelling Tumour Progression. Adv Exp Med Biol. 2016 899:231-44.
Wong J S, Cheah Y K. Potential miRNAs for miRNA-Based Therapeutics in Breast Cancer. Noncoding RNA. 2020 Jul. 13; 6 (3): 29.
Yin L, Duan J J, Bian X W, Yu S C. Triple-negative breast cancer molecular subtyping and treatment progress. Breast Cancer Res. 2020 Jun. 9; 22 (1): 61.
Zizza P, Dinami R, Porru M, Cingolani C, Salvati E, Rizzo A, D'Angelo C, Petti E, Amoreo C A, Mottolese M, Sperduti I, Chambery A, Russo R, Ostano P, Chiorino G, Blandino G, Sacconi A, Cherfils-Vicini J, Leonetti C, Gilson E, Biroccio A. TRF2 positively regulates SULF2 expression increasing VEGF-A release and activity in tumour microenvironment. Nucleic Acids Res. 2019 Apr. 23; 47 (7): 3365-3382.

1. A method of treating a tumor that overexpresses TRF2 in a patient, comprising administering a therapeutically effective amount of hsa-miR-182-3p to the patient.

2. The method according to claim 1, wherein said tumour overexpressing TRF2 is not osteosarcoma.

3. The method according to claim 1, wherein said tumour overexpressing TRF2 is not neuroblastoma.

4. The method according to claim 1, wherein said tumour is selected from breast cancer, cervical cancer, colon cancer, primary brain tumours, such as, for example, glioblastoma, metastatic brain tumours, pancreatic cancer, head and neck cancer.

5. The method according to claim 4, wherein said breast cancer is a triple negative breast cancer, for example a triple negative breast cancer mutated in BRCA1, optionally resistant to PARP inhibitors, or a HER2-positive breast cancer.

6. The method according to claim 1, wherein the hsa-miR-182-3p is encapsulated in lipid nanoparticles.

7. A method of treating a tumor that overexpresses TRF2 in a patient, comprising administering a therapeutically effective amount of a pharmaceutical composition comprising hsa-miR-182-3p, together with one or more excipients and/or adjuvants to said patient.

8. The method according to claim 7, wherein said tumour overexpressing TRF2 is not osteosarcoma.

9. The method according to claim 7, wherein said tumour overexpressing TRF2 is not neuroblastoma.

10. The method according to claim 7, wherein said tumour is selected from breast cancer, cervical cancer, colon cancer, primary brain tumours, such as, for example, glioblastoma, metastatic brain tumours, pancreatic cancer, head and neck cancer.

11. The method according to claim 10, wherein said breast cancer is a triple negative breast cancer, for example a triple negative breast cancer mutated in BRCA1, optionally resistant to PARP inhibitors, or a HER2-positive breast cancer.

12. The method according to claim 7, wherein the hsa-miR-182-3p is encapsulated in lipid nanoparticles.

13. The method according to claim 7, further comprising administering one or more antitumour drugs, such as, for example, platinum derivatives, such as Cisplatin, taxanes, such as Paclitaxel, pyrimidine analogues, such as Gemcitabine, anthracyclines, such as Doxorubicin, and immune checkpoint inhibitors, such as Atezolizumab, PARP1 inhibitors, such as Olaparib, and antibody-drug conjugates such as Sacituzumab-Govitecan to the patient.

14. The method according to claim 13, wherein the hsa-miR-182-3p and one or more antitumour drugs are administered separately or sequentially.

15. The method according to claim 14, wherein said tumour overexpressing TRF2 is not osteosarcoma.

16. The method according to claim 14, wherein said tumour overexpressing TRF2 is not neuroblastoma.

17. The method according to claim 14, wherein said tumour is selected from breast cancer, cervical cancer, colon cancer, and primary brain tumours, such as, for example, glioblastoma, metastatic brain tumours, pancreatic cancer, head and neck cancer.

18. The method according to claim 17, wherein said breast cancer is a triple negative breast cancer, for example a triple negative breast cancer mutated in BRCA1, optionally resistant to PARP inhibitors, or a HER2-positive breast cancer.

19. The method according to claim 14, wherein hsa-miR-182-3p is encapsulated in lipid nanoparticles.

20. A method of treating a patient with primary breast cancer who is at risk of developing metastasis, said method comprising measuring the expression of TRF2 in a sample of primary breast cancer from said patient,
wherein an expression level of TRF2 greater than the expression level of TRF2 in a sample of healthy tissue and/or in a sample of non-metastatic primary breast cancer indicates a risk of developing metastasis; and
administering a therapeutically effective amount an adjuvant therapy to said patient who is at risk of developing metastasis.
